(19) United States
(12) Patent Application Publication
(10) Pub. No.: US 2015/0165081 A1
(43) Pub. Date: Jun. 18, 2015

(54) METHOD FOR CONTROLLING STERILITY OF A STERILIZED MEDICAL DEVICE AND STERILIZATION MODULE FOR CARRYING OUT SAID METHOD

(71) Applicant: Peter KOZIN, Ljubljana (SI)

(72) Inventor: Peter Kozin, Ljubljana (SI)

(21) Appl. No.: 14/414,589

(22) PCT Filed: Jul. 18, 2013
(86) PCT No.: PCT/SI2013/000045

§ 371 Date: Jan. 13, 2015

(30) Foreign Application Priority Data
Jul. 18, 2012 (SI) 201200235

(51) Int. Cl. A61L 2/28; A61L 2/07; G01K 13/00; A61B 19/00

(52) U.S. Cl. CPC A61L 2/28; A61B 19/34; A61L 2/07; G01K 13/00

(57) ABSTRACT

In order to control sterility of a sterilized medical device after high-temperature steam sterilization, the medical device and a sterilization module are wrapped by a wrapper and put into an autoclave to carry out sterilization, during which the sterilization module logs a time history of its own temperature and pressure in its own environment. The sterilization module assesses the efficiency of sterilization by processing the acquired data. The sterilization module shifts from a normal mode to an alarm mode after an inefficient sterilization and/or after a sterility expiration date of the medical device is over. The sterilization module signals its mode upon each jerk. A sterility status of a uniquely identified medical device is unambiguously and simply presented to a user by a lighting-up of a single light source on the sterilization module that is suitable for a routine control of the efficiency of sterilization, and is reusable.

The invention relates to a method for controlling sterility of a sterilized medical device after high-temperature steam sterilization, wherein the medical device is placed close to a sterilization module, they are both wrapped by a sterilization wrapper and put into an autoclave to carry out sterilization. The invention also relates to a sterilization module intended for controlling sterility of a sterilized medical device and for carrying out said method. The module is provided with a communication circuit. The sterilization module is foreseen for a routine control of efficiency of sterilization and is reusable.

Controlling of efficiency of sterilization of medical devices, e.g. surgical instruments, by means of high-temperature steam in an autoclave is known. Said controlling is based on determination of colour of an indicator label, which is arranged on the medical device prior to sterilization and they are both wrapped by a sterilization wrapper. The indicator label is disposable. Its colour indicates the extent, to which a chemical reaction therein occured. Said extent is characteristic of a period of maintenance at a prescribed high temperature. On the one hand, uncertainty of assessed efficiency of sterilizing is quite high, on the other hand, the sterilization wrapper needs to be unwrapped before assessing the status of the indicator label. This is only done directly prior to use of a medical device. Due to the mentioned drawbacks, such controlling of efficiency of sterilization only has a limited application in sterilization of medical devices.

U.S. Pat. No. 7,122,150 B2 discloses an electronic reader, which is wrapped into a sterilization wrapper together with a medical device. A disposable label is used as a sterilization indicator. It is inserted into said reader and the colour of the illuminated label after sterilization is read out. The read-out value is evaluated in an interpretation circuit of the reader. The reader determines efficiency of the performed sterilization and the evaluation result is wirelessly signalled to an interrogator by means of a reader communication circuit. Indeed the efficiency of the performed sterilization is determined before the sterilization wrapper is removed and the reader is reusable as well, but said very high uncertainty of measurement still remains due to integral sensing of development during sterilization only by the colour of the indicator label as the only recorded parameter.

Good practice in the field of hospital sterilization of medical devices as well as standards ISO EN 11607 and ISO EN 17665 require that sterilization should be carried out in a controlled, reproducible and traceable way.

The technical problem to be solved by the present invention is to propose a method, which should provide for automatical determination of efficiency of performed sterilization by logging a time history of the temperature of a medical device as well as the pressure in its environment during sterilization and when a sterilization package is unwrapped directly prior to using a medical device, information on sterilization efficiency determined in said way and on potential sterility expiration date should be conveyed to a user and additionally, an operator of an sterilization machine should be able to make an ex post analysis of time history from the beginning of the sterilization of the medical device onwards. A further task of the invention is to propose a module for carrying said method.

The technical problem of the invention is solved by the method of the invention intended for controlling sterility of a sterilized medical device as characterized by the features of claim 1 and by the sterilization module for controlling sterility of a sterilized medical device as characterized by the features of claim 10. Dependent claims characterize variants of their embodiments.

The technical solution as proposed by the invention primarily stands out for the following advantages. The sterility status of a uniquely identified medical device is unambiguously and simply presented to a user by the fact that a single light source on the sterilization module lights up. The sterilization module is suitable for a routine control of efficiency of sterilization and is reusable.

Immediately after sterilization is over, its efficiency is assessed on the basis of time history of temperature and pressure that were electronically acquired by the sterilization module. An analysis of the values of temperature, pressure and other physical quantities during sterilization is made possible as well

The invention will now be explained in more detail by way of a description of an embodiment and variants of the method of the invention for controlling sterility of a sterilized medical device as well as a sterilization module of the invention and with reference to the accompanying drawing representing in

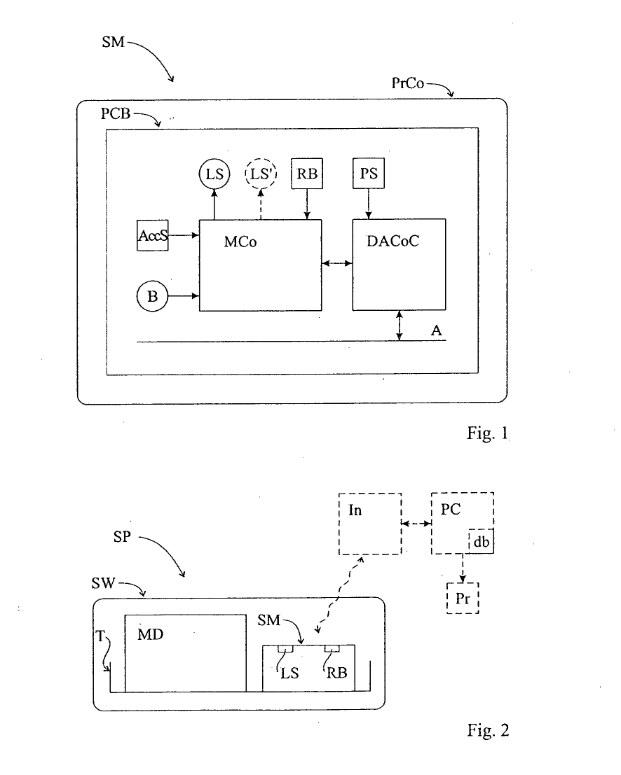

FIG. 1 a block diagram of the sterilization module of the invention for controlling sterility of a sterilized medical device, and

FIG. 2 a sterilization package comprising the sterilization module of the invention and the medical device as well as an interrogator and a computer for carrying out further advantageous processing steps after sterilization is completed.

A method of the invention for controlling sterility of a sterilized medical device MD is foreseen to be used in high-temperature steam sterilization. In a known way, the medical device MD is placed on a tray T close to a sterilization module SM, and they are wrapped by a sterilization wrapper SW (FIG. 2). A sterilization package SP is put into an autoclave to carry out steam sterilization.

Prior to first use, the sterilization module SM is labelled by entering its identification mark into its memory.

According to the invention, the sterilization module SM is reset before sterilization is initiated. The sterilization module SM thus gets initialized and activated. Before the sterilization module SM together with the medical device MD are wrapped by the sterilization wrapper SW, a reset button RB is pressed, for example, to reset the sterilization module SM (FIG. 1).

According to the invention, the sterilization module SM logs a time history of the temperature within the sterilization package SP, i.e. the temperature of the medical device MD.

During the sterilization the sterilization module SM logs a time history of pressure within the sterilization package SP as well.

According to the invention upon completion of sterilization, the sterilization module SM automatically processes the acquired data and assesses the efficiency of sterilization.

According to the invention, the sterilization module SM shifts from a normal mode to an alarm mode if it is determined during said data processing that the sterilization was inefficient or that sterility expiration date of the medical device MD expired later.

The sterility expiration date is calculated according to the standards in force, namely from completion of sterilization onwards. During sterilization, the sterilization module SM therefore detects a drop in temperature below the prescribed temperature value as sterilization completion.

According to the invention, the sterilization module SM keeps transmitting a signal on its mode for several seconds upon each jerk.

It is accordingly proposed by the invention that, in an advantageous way, a single light source LS on the sterilization module SM signals a basic signal on the mode of the sterilization module SM.

The sterilization module SM is in a normal mode if the medical device MD is sterile, which means that the sterilization was efficient and the sterility expiration date of the medical device MD is not yet over. The sterilization module SM is in an alarm mode if sterilization was inefficient or the sterility expiration date of the medical device MD is over after an efficient sterilization.

As a variant embodiment of the method of the invention it is proposed that, by their way of turning on, light sources LS, LS′, . . . —variously coloured light emitting diodes may be used—on the sterilization module SM signal data on efficiency of performed sterilization and on sterility expiration date of the medical device MD non being over as well as on other characteristic features of the performed sterilization.

In an advantageous way, the sterilization module SM automatically signals data on sterilization and sterility of the medical device MD to an interrogator In when it is moved to its close proximity. Then the interrogator In transmits said data to a personal computer PC. A printer Pr can then print the data from a computer database db onto a partly two-layered label. The first part of said label is stuck onto the sterilization wrapper SW comprising the sterilized medical device MD and the sterilization module SM, and the second part of the label is stuck onto a patient file directly prior to use of the sterilized medical device MD.

The invention further proposes, a sterilization module SM for controlling sterility of a sterilized medical device MD. The sterilization module SM is equipped for radio communication.

According to the invention, the sterilization module SM is provided with a reset circuit that initializes and activates the sterilization module SM before sterilization is initiated. The sterilization module SM is for instance provided with a reset button RB for resetting that is used before the sterilization module SM together with the medical device MD are wrapped by the sterilization wrapper SW.

The sterilization module SM of the invention is further provided with a data acquisition and communication circuit DACoC. Said circuit, e.g. IDS-SL900A, comprises at least one temperature sensor, a pressure sensor PS may be connected thereto. The data acquisition and communication circuit DACoC is connected to an antenna A.

According to the invention, the sterilization module SM is provided with a microcontroller MCo connected to the data acquisition and communication circuit DACoC. The microcontroller MCo automatically processes the acquired data immediately after completion of sterilization and maintains the sterilization module SM in a normal operation mode or shifts it to an alarm mode depending on efficiency of the performed sterilization and/or on the expired sterility date of the medical device MD.

The sterilization module SM of the invention is further provided with an acceleration sensor AccS connected to an input of the microcontroller MCo. Upon each jerk of the sterilization module SM the acceleration sensor AccS generates a signal that triggers the microcontroller MCo to keep signalling the mode of the sterilization module SM for several seconds.

The sterilization module SM is also provided with a light source LS connected to an input of the microcontroller MCo. Depending on the sterilization module mode, the light source LS signals either a signal of sterility of the medical device MD or a signal of the alarm mode. The light source LS keeps transmitting said signal for several seconds.

The sterilization module SM may be provided with several light sources LS, LS′, . . . , which may be variously coloured and are connected to inputs of the microcontroller MCo. By their way of turning on, they signal the data on the efficiency of the performed sterilization and/or on the sterility expiration date of the medical device MD being over as well as on other characteristic features of the performed sterilization.

Light emitting diodes are used as light sources LS, LS′, . . .

A battery B supplies a circuit of the sterilization module SM. Said circuit is fabricated on a printed circuit board PCB having dimensions such as 4 cm×9 cm×1 cm. The printed circuit board PCB is coated by a thin polymer protective coating PcCo. All components of the sterilization module SM are resistant to high temperature existing during steam sterilization.

The database db of data on the latest sterilization as well as on previous sterilizations performed by said sterilization module SM is created in the personal computer PC. The personal computer PC is connected through an interrogator In with the sterilization module SM, wherefrom it receives the acquired data. The data for printing a partly two-layered label for the sterilized medical device MD after sterilization are acquired from said database db. One part of said label is stuck onto the sterilization package SP, which comprises the medical device MD and the sterilization module SM, and second part thereof is stuck onto a patient file.

1. A method for controlling sterility of a sterilized medical device after a high-temperature steam sterilization,
wherein the medical device is placed close to a sterilization module, they are both wrapped by a sterilization wrapper and
put into an autoclave to carry out sterilization,
characterized in
that the sterilization module is reset before sterilization is initiated, that the sterilization module logs a time history of
its own temperature and pressure in its own environment
during sterilization,
that the sterilization module assesses the efficiency of sterilization by processing the acquired data,
that the sterilization module shifts from a normal mode to an alarm mode after an inefficient sterilization and/or
after a sterility expiration date of the medical device is over
and that the sterilization module signals its mode upon each jerk.

2. The method as recited in claim 1, characterized in
that the sterilization module is reset by pressing a reset button
before the sterilization module together with the medical device is wrapped by the sterilization wrapper.

3. The method as recited in claim 1, characterized in
that the sterilization module logs a drop in temperature during sterilization below a specified value as completion of sterilization.

4. The method as recited in claim 1, characterized in
that, depending on the mode of the sterilization module,
a light source on the sterilization module signals
either sterility of the medical device
or the alarm mode.

5. The method according to claim 1, characterized in
that, by their way of turning on, variously coloured light sources on the sterilization module signal the data on
the efficiency of the performed sterilization and
sterility of the medical device.

6. The method as recited in claim 4, characterized in
that a light emitting diode is used as the light source.

7. The method according to claim 1, characterized in
that the wrapped sterilization module signals the data on
the sterilization time history and
sterility of the medical device
to an interrogator when moved to close proximity of said interrogator.

8. The method as recited in claim 7, characterized in that the interrogator transfers said data on the sterilization time history to a computer.

9. The method as recited in claim 8, characterized in
that a printer prints key data from a computer database onto a partly two-layered label, a first part of which label is stuck onto the sterilization package,
which comprises the sterilized medical device and the sterilization module, and a second part of which is stuck onto a patient file
prior to use of the sterilized medical device.

10. A sterilization module (SM) for controlling sterility of a sterilized medical device provided with a communication circuit,
characterized in
that it is provided with a reset circuit,
which initializes and activates the sterilization module (SM)
before initiation of sterilization,
that it is provided with a data acquisition and communication circuit (DACoC) provided with at least one temperature sensor and
whereto a pressure sensor (PS) is connected,
that it is provided with a microcontroller (MCo)
connected to the data acquisition and communication circuit (DACoC) and processing the data acquired during sterilization and
shifting from a normal mode to an alarm mode
at an inefficient sterilization and/or
after a sterility expiration date of the medical device is over
and that it is provided with an acceleration sensor (AccS)
connected to an input of the microcontroller (MCo) and
triggering it for the sterilization module (SM) to signal its mode upon each jerk.

11. The sterilization module (SM) as recited in claim 10, characterized in
that it is provided with a reset button (RB) for resetting the sterilization module (SM) to be initialized and activated hereby
before the sterilization module (SM) together with the medical device
is wrapped by the sterilization wrapper.

12. The sterilization module (SM) as recited in claim 10, characterized in that an output of the microcontroller (MCo) is connected to a light source (LS) that, depending on the sterilization module mode, signals
either sterility of the medical device
or the alarm mode.

13. The sterilization module (SM) as recited in claim 10, characterized in
that several outputs of the microcontroller (MCo) are connected to
variously coloured light sources (LS, LS′, . . . )
that, by the way of their turning on,
signal data on the efficiency of the performed sterilization and
signal an alarm mode
after an inefficient sterilization and/or
after a sterility expiration date of the medical device is over.

14. The sterilization module (SM) as recited in claim 11,
characterized in
that light emitting diodes are used as light sources (LS, LS′, . . . ).

15. The sterilization module (SM) as recited in claim 13, characterized in that the sterilization module (SM) transfers the processed data on the performed sterilization by means of an interrogator (In), when they are close to each other, to a personal computer (PC),
wherein a base (db) of data on sterilizations is created,
in which the sterilization module (SM) was used.

16. The sterilization module (SM) as recited in claim 13, characterized in that a two-part label is printed for the sterilized medical device,
one part of which is stuck onto the sterilization package
comprising the medical device and the sterilization module (SM), and
a second part of which is stuck onto a patient label.

17. The sterilization module (SM) as recited in claim 15, characterized in that the sterilization module (SM) is fabricated as a printed circuit board (PCB) coated by a thin polymer protective coating (PcCo).

18. The sterilization module (SM) as recited in claim 17, characterized in that its components are resistant to high temperature during steam sterilization.
